(19) United States
(12) Patent Application Publication
(10) Pub. No.: US 2017/0367583 A1
(43) Pub. Date: Dec. 28, 2017

(54) METHODS AND SYSTEMS FOR PERFORMING TISSUE CLASSIFICATION USING MULTI-CHANNEL TR-LIFS AND MULTIVARIATE ANALYSIS

(71) Applicant: Cedars-Sinai Medical Center, Los Angeles, CA (US)

(72) Inventors: Keith L. Black, Los Angeles, CA (US); Michael Baker, Portland, OR (US); Howland Jones, Rio Rancho, NM (US); Pramod Butte, Los Angeles, CA (US)

(21) Appl. No.: 15/683,277

(22) Filed: Aug. 22, 2017

Related U.S. Application Data
(63) Continuation of application No. PCT/US2016/059054, filed on Oct. 27, 2016.
(60) Provisional application No. 62/248,934, filed on Oct. 30, 2015.

(51) Int. Cl. A61B 5/00; G06K 9/62; A61B 10/02; G06T 7/00; G01N 21/64; A61B 5/08

(52) U.S. Cl. CPC A61B 5/0071; G01N 21/6408; A61B 5/08; A61B 5/4064; A61B 5/4255; A61B 5/441; A61B 5/7267; A61B 10/02; G01N 21/6402; G06T 7/0012; G06K 9/6267; A61B 5/0091; A61B 2576/026; G01N 2201/06113; G06T 2207/10064; G06T 2207/30016; G06T 2207/30028; G06T 2207/30061; G06T 2207/30068; G06T 2207/30088

(57) ABSTRACT

Described herein are methods and systems for analyzing a sample by applying time resolved laser induced fluorescence spectroscopy to the sample to measure lifetime time decay profile data relating to the sample, and applying multivariate analysis to process the data so as to classify a sample as, for example, normal or abnormal. The sample may be cells, fluid or tissue from any organ. The sample may be in vitro or in vivo. The data may be obtained in situ or in vitro.

### CROSS-REFERENCE

This application is a continuation application of Serial No. PCT/US2016/059054 (Attorney Docket No. 49620-703.601), filed Oct. 27, 2016, which is a non-provisional of, and claims the benefit of U.S. Provisional Application No. 62/248,934 (Attorney Docket No. 49620-703.101), filed Oct. 30, 2015; the entire contents of each of the above listed patent applications are incorporated herein by reference.

### TECHNICAL FIELD

Provided herein are methods and systems for classifying a sample, including distinguishing normal sample from abnormal sample by obtaining data using Time Resolved Laser Induced Fluorescence Spectroscopy (TR-LIFS) and processing the data using multivariate analysis as described herein.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

It is highly desirable to be able to identify tissue types and boundaries, for example when attempting to remove malignant tissue from a patient. Traditionally, this may be a very time consuming and cumbersome process, potentially requiring tissue to be removed and subjected to follow up laboratory examination to determine tissue type(s).

For example, surgical operations to remove cancerous tissue may require a variety of pre-surgical imaging and/or marking to estimate tissue boundaries, intentional removal of suspect or excess tissue during surgery, and then follow up laboratory testing of the removed tissue to determine if the surgery successfully removed the undesired tissue. Thus, some guesswork is involved in critical surgical operations, such as brain surgery, where time is at a premium and precise margin detection (to minimize removal of normal tissue) is highly desirable, but the cost of potentially leaving malignant tissue in the patient is also extremely high.

To improve this process, the inventors have developed a process to interrogate tissue in the body during surgery. Because no rigorous processing techniques are needed before performing the analysis, and the tissue does not need to be removed from the patient to be analyzed, the classification process can take place in near real-time during a surgical operation. Thus, patient outcomes may be significantly improved, and surgical time and cost may be substantially reduced.

### SUMMARY

The following embodiments and aspects thereof are described and illustrated in conjunction with systems, compositions and methods which are meant to be exemplary and illustrative, not limiting in scope.

According to aspects of the present disclosure, a method for analysis of tissue is provided. According to the method, time resolved laser induced fluorescence spectroscopy is applied to a tissue, and lifetime time decay profile data relating to the tissue is measured at several specific emission wavelength bands. The lifetime decay profile data is normalized for each of the specific emission wavelength bands, and the data is concatenated to generate a multi-channel fluorescence decay response curve. Multivariate curve resolution is applied to the multi-channel fluorescence decay response curve to generate a plurality of decay response signature components and corresponding intensity data. A biopsy of the tissue is performed, and the biopsy information and the intensity data are used to determine a tissue classification type indicated by the intensity data.

According to further aspects of the present disclosure, a system for diagnosis of human tissue is disclosed, the system having a database, a scope, and a processor. The database contains human tissue data for a variety of tissue classification types along with a plurality of decay profile signatures and corresponding intensities. The scope collects time resolved laser induced flourescense spectropscopy data from a human tissue. The processor receives the time resolved laser induced flourescense spectropscopy data from the scope, and determines lifetime decay profile data. The processor generates decay profile signature data and corresponding intensity data based on the lifetime decay profile data, and communicates with the database to identify the classification type of the tissue according to the intensity data.

According to further aspects of the present disclosure, a method for identifying human tissue according to spectral information is provided. The method uses a computing system with one or more processors in communication with a network database. According to the method, time resolved laser induced fluorescence spectroscopy is applied to a human tissue, and lifetime time decay profile data relating to the human tissue is measured at several specific emission wavelength bands. The lifetime decay profile data is normalized for each of the specific emission wavelength bands, and the data is concatenated to generate a multi-channel fluorescence decay response curve. The one or more processors are used to apply a curve fitting technique to the generated multi-channel fluorescence decay response curve, to determine intensity data corresponding to a plurality of decay response signature components. The one or more processors send a request, including information relating to at least one of the plurality of decay response signature components and corresponding intensity data, to the network database to identify the human tissue. The one or more processors receive a response from the network database, indicating the tissue classification type of the human tissue per the intensity data.

According to further aspects of the present disclosure, a method for classifying samples according to intensity data is provided. According to the method, time resolved laser induced fluorescence spectroscopy is applied to a sample of known type, lifetime time decay profile data relating to the sample is measured at specific emission wavelength bands, the lifetime time decay profile data is normalized for each specific emission wavelength band, and concatenated to generate a multi-channel fluorescence decay response curve. The above steps are repeated for additional samples of known type, and a combined data set is generated from the multi-channel fluorescence decay response curve for each sample. Multivariate curve resolution is applied to the combined data set, generating decay response signature components, and intensity data corresponding to each sample. Using the intensity data and the known sample types, a classification model is determined.

These and other capabilities of the disclosure will be more fully understood after a review of the following figures, detailed description, and claims.

### BRIEF DESCRIPTION OF THE DRAWINGS

Exemplary embodiments are illustrated in referenced figures. It is intended that the embodiments and figures disclosed herein are to be considered illustrative rather than restrictive.

FIG. 1 illustrates a process for analyzing and classifying tissue, according to an embodiment of the present invention.

FIG. 2 illustrates an averaged measurement containing the decay response curves for a set of 6 wavelength bins, according to an embodiment of the present invention.

FIG. 3 illustrates the decay response curves after preprocessing, according to an embodiment of the present invention.

FIG. 4 illustrates a combination of multi-channel fluorescence decay response curves from data for 35 measurements, according to an embodiment of the present invention.

FIG. 5 illustrates three multivariate curve resolution (MCR) starting components obtained by averaging like-type tissue measurements together, according to an embodiment of the present invention.

FIG. 6 illustrates saw-tooth noise components corresponding to a set of six wavelength bins, according to an embodiment of the present invention.

FIG. 7 illustrates additional MCR components obtained upon initialization using random numbers, according to an embodiment of the present invention.

FIG. 8 illustrates final MCR decay response components for the three brain tissue types, according to an embodiment of the present invention.

FIG. 9 illustrates additional MCR components that model additional measurement variance present in the multi-channel decay response data, according to an embodiment of the present invention.

FIG. 10 illustrates corresponding intensity values for three brain tissue types, according to an embodiment of the present invention.

FIG. 11 illustrates the reconstruction of a multi-channel response measurement using MCR decay response components (top) and residual data (bottom), according to an embodiment of the present invention.

FIG. 12 illustrates a flow diagram of steps used to classify brain tissue using TR-LIFS data, according to an embodiment of the present invention.

While the invention is susceptible to various modifications and alternative forms, specific embodiments have been shown by way of example in the drawings and will be described in detail herein. It should be understood, however, that the invention is not intended to be limited to the particular forms disclosed. Rather, the invention is to cover all modifications, equivalents, and alternatives falling within the spirit and scope of the invention as defined by the appended claims.

### DETAILED DESCRIPTION

All references cited herein are incorporated by reference in their entirety as though fully set forth. Unless defined otherwise, technical and scientific terms used herein have the same meaning as commonly understood by one of ordinary skill in the art to which this invention belongs. Allen et al., Remington: The Science and Practice of Pharmacy 22nd ed., Pharmaceutical Press (Sep. 15, 2012); Hornyak et al., Introduction to Nanoscience and Nanotechnology, CRC Press (2008); Singleton and Sainsbury, Dictionary of Microbiology and Molecular Biology 3rd ed., revised ed., J. Wiley & Sons (New York, N.Y. 2006); Smith, March's Advanced Organic Chemistry Reactions, Mechanisms and Structure 7th ed., J. Wiley & Sons (New York, N.Y. 2013); Singleton, Dictionary of DNA and Genome Technology 3rd ed., Wiley-Blackwell (Nov. 28, 2012); and Green and Sambrook, Molecular Cloning: A Laboratory Manual 4th ed., Cold Spring Harbor Laboratory Press (Cold Spring Harbor, N.Y. 2012), provide one skilled in the art with a general guide to many of the terms used in the present application. For references on how to prepare antibodies, see Greenfield, Antibodies A Laboratory Manual 2nd ed., Cold Spring Harbor Press (Cold Spring Harbor N.Y., 2013); Köhler and Milstein, Derivation of specific antibody-producing tissue culture and tumor lines by cell fusion, Eur. J. Immunol. 1976 July, 6(7):511-9; Queen and Selick, Humanized immunoglobulins, U.S. Pat. No. 5,585,089 (1996 December); and Riechmann et al., Reshaping human antibodies for therapy, Nature 1988 Mar. 24, 332(6162):323-7.

One skilled in the art will recognize many methods and materials similar or equivalent to those described herein, which could be used in the practice of the present invention. Other features and advantages of the invention will become apparent from the following detailed description, taken in conjunction with the accompanying drawings, which illustrate, by way of example, various features of embodiments of the invention. Indeed, the present invention is in no way limited to the methods and materials described. For convenience, certain terms employed herein, in the specification, examples and appended claims are collected here.

Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs.

The disclosures herein detail the application of multivariate analysis techniques to Time Resolved Laser Induced Fluorescence Spectroscopy (TR-LIFS) data in order to classify tissue, and predict tissue type of future samples. The procedure was developed using a fluorescence lifetime measurement capable of interrogating tissue in the brain during surgery, although additional biological cells, fluids and tissues could be classified with the same technique. Since fluorescence species have a unique time decay profile, these fluorescence lifetime decay measurements can be analyzed to identify component signatures and corresponding intensities, and subsequently used to guide the surgeon and identify tissue types and tissue boundaries. According to some embodiments, the process is applied during brain surgery to identify tissue types (for example, normal cortex, white matter, and glioblastoma) and tissue boundaries present in the brain.

Preferably, the fluorescence lifetime decay profiles are measured at a specific set of emission wavelengths. According to some embodiments, multiple sets of emission wavelengths are used to gather unique decay profiles for each sample, as the use of several decay profiles can provide additional specificity in classifying different tissues due to the combination of the unique decay profiles. For example, six decay profiles may be gathered for each sample by using six separate wavelength bins for emission.

Multivariate analysis techniques traditionally used to analyze spectroscopic and hyperspectral image data sets can then be used to develop a classification system that simultaneously utilizes all of these decay profiles. One technique known as Multivariate Curve Resolution (MCR), is especially well suited for obtaining unknown spectral signatures. By using a training set of known tissue samples, the spectral signatures for the tissue types can be identified, and then applied to one or more additional samples to classify or predict the tissue type(s) of the additional samples. Better quality results may be obtained if the training set comprises multiple measurements for each tissue type of interest, and each tissue type is collected from multiple subjects, allowing the analysis to account for variations due to independent, non-tissue measurement variances (such as instrument artifacts, noise, or physiological factors). According to some embodiments, the training set comprises at least ten measurements for each tissue type of interest, and each tissue type is collected from at least three separate subjects. Once the spectral signatures are determined from the training set, these signatures may be applied to future sample sets by using simpler algorithms such as Classical Least Squares (CLS) [3,4], in which the spectral signatures are projected onto the new sample data to obtain the intensities of each signature for each sample. The intensity information is then used to classify the tissue by type.

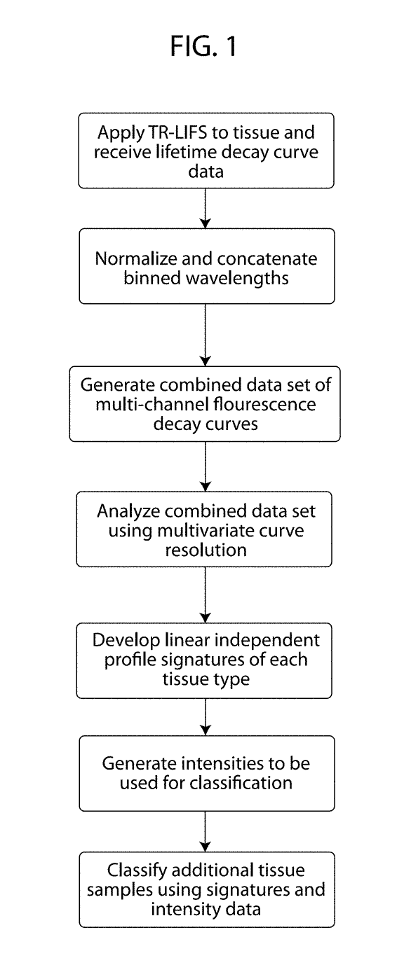

Turning to FIG. 1, a process for analysis of fluorescence emissions and classification of tissue is described. Each element of the process is discussed in detail below.

### Receive Lifetime Decay Curve Data

The process begins by interrogating the tissue of interest using the TR-LIFS process (see PCT/US2014/030610, published as WO 2014/145786). The lifetime decay curve information for a tissue can be measured by exciting the tissue region with a pulsed laser, and collecting the fluorescence emission in a time-resolved manner. See [1]. The fluorescence emission, depending on the excited endogenous fluorophores, has a decay lifetime specific to the fluorophore. The goal is to use these lifetime measurements to discriminate between distinct but important tissue types. Emission lifetime decay curve data may be collected at multiple wavelength ranges (referred to herein as wavelength “bins”) to achieve a more detailed data set. Because excited fluorophores, specific to the tissue types of interest, may have more intense emissions at different wavelengths, collecting these fluorescence decay curves over several different wavelength bins should allow tissue discrimination to be more specific.

### Normalize and Concatenate Binned Wavelengths

Before applying Multivariate Curve Resolution (MCR) to determine the best fit for the raw decay response curve data, some preprocessing of the data may be performed to improve the accuracy of the analysis. The preprocessing of the data consists of the following, although not all preprocessing treatments are required and alternative embodiments may use only a portion of the preprocessing treatments.

According to some embodiments, the measurement of the fluorescence decay response is performed many times (for example, 1000 repetitions), and the measurement data is then averaged together to improve the overall signal to noise of the measurement.

Since the critical information is contained in the decay responses of the wavelength bins, the data set can be reduced to focus on the temporal data points near the peak of each decay curve. For example, according to some embodiments, the 10 temporal data points immediately prior to the start of a peak are included, and the next 100 data points immediately thereafter are included, and the other data points are truncated. By focusing the data on the critical information regions, the overall data set is reduced and therefore processing speed is improved.

Additionally, due to changes in laser intensities across measurements, the absolute overall intensity information may be unreliable. Therefore, according to some embodiments, the overall intensity is adjusted and normalized on a per decay curve basis to compensate for effects of laser intensity and/or other instrumental changes. After the truncation of each decay curve, as detailed above, the minimum value of each decay curve is subtracted from the entire curve and then normalized by dividing by the maximum intensity.

At the conclusion of the treatments above, or a portion thereof, the decay curves are then concatenated back together to provide multi-channel fluorescence decay response curves for each measurement. Multi-channel refers to the several binned fluorescence emission channels.

Generate a Combined Data Set from the Multi-Channel Fluorescence Decay Response Curves for Each Measurement

Next, the multi-channel fluorescence decay response data is combined together, so that the data can be analyzed using MCR to identify the differences in all wavelength decay responses with respect to the tissue types of interest.

Although not required, it is preferable to perform multiple measurements, using multiple samples, and therefore generate multiple multi-channel fluorescence decay response curves for each tissue type of interest. By doing so, this potentially reduces the effect of any measurement error or variance associated with an individual sample in the training set.

From the combined data set for the training samples, MCR can be applied to determine the independent spectral signatures associated with the tissue types of interest, as discussed below.

### Analyze Combined Data Set Using MCR

MCR has been used in fluorescence hyperspectral imaging to discover all independently varying fluorescence species above the noise (spectral signatures and corresponding intensities of each signature) within an image without any a-priori information about the sample [2,3]. In this case, the starting components for the MCR analysis are initialized using a string of random numbers. However, the preferred case will be that a known training set of tissue types will be used for the initial analysis, where the tissue types for the samples in the training set have been confirmed by biopsy or other medical process of confirmation. In this case, the starting components can be initialized using an average value for each known tissue type, allowing the MCR analysis to modify the initial starting components to best fit the training data.

Once these fluorescence species or spectral signatures are obtained from a training set of samples, these pure component signatures can be applied to future sample sets by using simpler algorithms such as Classical Least Squares (CLS) [4-7], in which the pure spectral component signatures are projected onto the new sample data to obtain the intensities of each component for each sample. The intensities generated by either the MCR or CLS algorithms can then be used for sample classification.

The TR-LIFS data provides lifetime decay profiles which have unique signatures depending on the interrogated tissue sample. MCR is capable of extracting the unique signatures associated with these decay profiles. MCR can be applied to develop a set of pure decay response components associated, and not associated, with the tissue types. When doing so, it is preferred to account for both the desired components (components directly related to the tissue of interest) and components associated with interferences (noise, imprecision in the time zero peak location, etc.). If both are not accounted for properly, then the resulting sensitivity and specificity of the classification model can be poorer.

MCR is an alternating least squares fit of the data. Assume a linear additive data set D.

D=LC+E Equation 1:

where D is an m×n multi-channel decay response matrix, where m is the number of temporal decay data points and n is the number of measurements in the data. K is an m×p matrix of pure decay response components (signatures), where p is the number of pure decay response components. C is a p×n matrix of the intensities for each decay response component and each measurement. E is an m×n spectral matrix of unmodeled decay response variances (decay residuals) that are not accounted for within the MCR model. It's essentially the resulting error in the MCR modeling process. There is instrumental noise contained within the decay residual, therefore it is important to characterize the instrument noise and minimize the noise (if possible). Noise is generally considered anything that is not related to the pure decay response components of interest.

For example, if it is known that the data is composed of 3 components (corresponding to 3 tissue types of interest), then a single decay response measurement (d) can be described using equation 1a. Essentially it is the summation of the component shape (k) times the amount of that shape (c) for each component plus any uncertainties or noise (e), where each (k) is a m×1 vector and each (c) is the corresponding scalar quantity of each (k).

d₁=k₁c₁+k₂c₂+k₃c₃+e Equation 1a:

MCR is a constrained alternating least square method that allows one to solve for the intensities (equation 2) using estimates of the starting decay response components. Then these new intensity estimates are used to estimate new pure decay response components (equation 3). This alternating process, solving for either C or K, is continued until the C and K estimates no longer change substantially and convergence has been reached. When the analysis has converged to a solution, it provides the decay response components and their corresponding intensities for each measurement.

Ĉ=\hat{K}^{T}(\hat{K}\hat{K}^{T})⁻¹D Equation 2:

where \hat{K}^{T}(\hat{K}\hat{K}^{T})⁻¹ is the pseudo-inverse of the pure component matrix K

\hat{K}=DĈ^{T}(ĈĈ^{T})⁻¹ Equation 3:

where Ĉ^{T}(ĈĈ^{T})⁻¹ is the pseudo-inverse of the intensities matrix C

According to some embodiments, convergence is aided through constraints placed upon the MCR analysis. The most commonly employed constraint is the non-negativity constraint which prevents the components and intensities from going negative. See also [2] (discussing non-negativity constraints). Other constraints that can be placed upon the analysis are called equality constraints. These constraints prevent components from changing. Therefore, if a component is known, and should be fixed to its known value, an equality constraint holds the component while allowing MCR to change the other components present in the data, such that the overall residuals (E) are minimized.

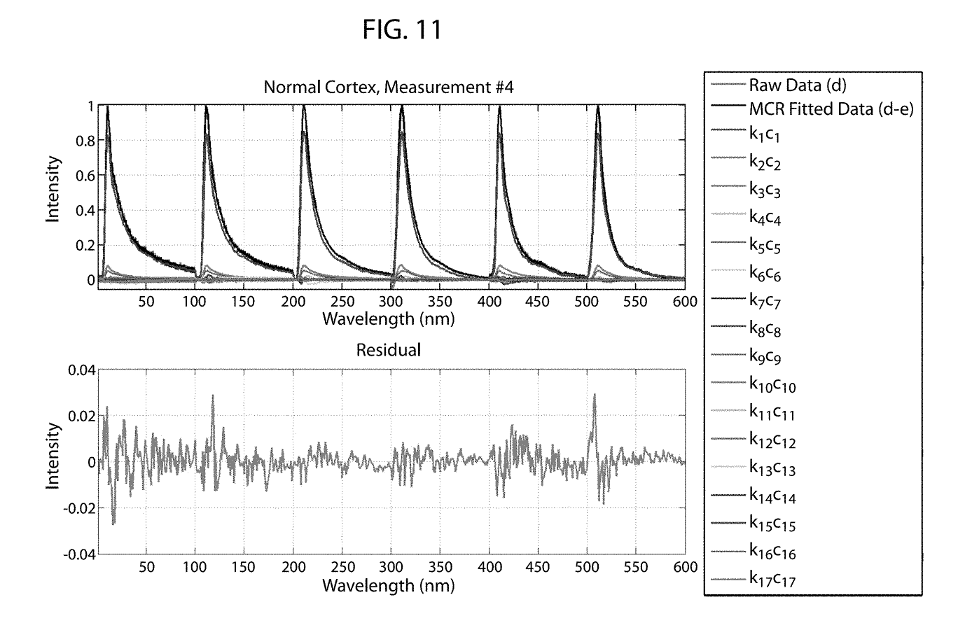

As described earlier, initial estimates of the decay response components are necessary to begin the MCR analysis. These initial estimates can be from previous analyses, random numbers, or can be obtained using knowledge about the data set itself. It is also necessary to determine how many components to use in the MCR analysis. One method of determining the preferred number of components is using a principal component analysis (PCA) Scree plot and identifying the number of eigenvalues above the noise floor. According to some embodiments, it is desirable to model known noise in the data using one or more components. Additionally, it may be desirable to analyze the residual data from the MCR analysis (see, e.g., FIG. 11) using PCA to determine whether any major signatures were not modeled, and if so, the number of components may be modified and MCR can be re-applied.

### Identify MCR Decay Response Components for the Tissue Types of Interest

The application of MCR develops the linear independent decay response components for each tissue type and their corresponding intensities. In addition, MCR models the other components that account for noise and other measurement variations (peak location, baseline variation, etc.). By modeling all the decay response variance (desired signal and noise), the sensitivity and specificity in the classification is improved. Alternatively, if only the main signal components are used to account for all variances present in the data, then the MCR method will use only the signal components to minimize the overall residuals when modeling the data, and therefore these signal components are fitting non-signal related variance, which will yield poorer intensity (C) estimates. The intensity estimates are important as they are used to classify between the tissue types, thus, for best results, it is preferable to model the noise component(s) as part of the MCR analysis.

### Generate Intensities Used for Classification and Classify Tissue Types

The intensity values (C) for each tissue sample is generated by MCR or CLS using equation 2 above. Following the MCR iterative least squares process, both the pure decay response components (signatures) and the amount of these components (intensities) are generated. CLS will use the same pure decay response components (as initially generated by MCR) and apply equation 2 to generate the intensities (C) used for classification.

The intensity values for each of the main (non-noise) components can then be used to classify each sample in the training set. Only the intensity values associated with the main components are required for classification, intensity related to noise or other artifacts may be ignored for purposes of classification.

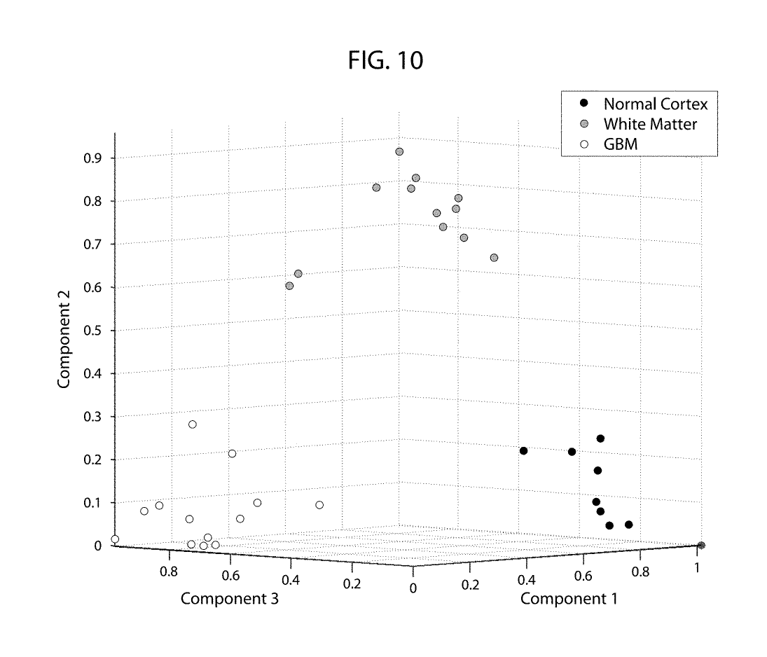

For example, if there are 3 main components, the intensities of each component may be determined per equation 2, and charted as in FIG. 10 for each sample in the training set. The intensity data for each of the main components will facilitate the classification of the tissue type based on the grouping, or class separation, shown in the data. For example, FIG. 10 shows a clear grouping by intensity values of normal cortex, white matter, and glioblastoma tissues.

Using these intensity values, and the grouping of the known tissue samples of the training set, a discriminate classification model may be prepared. Examples of discriminate analysis methodologies include: 1) Linear Discriminate Analysis (LDA), 2) Quadratic Discriminate Analysis (QDA) or 3) using Mahalanobis distance to discriminate. Other models may also be used to perform the tissue classification as appropriate for a particular intensity data set. The discriminate model may then be applied to classify additional tissue samples by using the intensity values of the main components, as detailed in the following section. This discriminate model can be used to classify future tissue samples according to intensities obtained from the MCR, CLS, or ACLS analysis techniques using the same main decay response components. If, however, additional tissue type(s) are introduced to the process, then a new model must be developed using an appropriate training set.

According to some embodiments, if additional samples are measured and tested (e.g., by biopsy or other verification method) the measurements can be added to the training set, and the discriminate model can be adjusted accordingly. The addition of additional verified samples may improve the MCR estimate of the multi-channel decay components, and lead to even tighter groupings by intensity value.

### Classifying Additional Tissue Using CLS, ACLS or MCR

As discussed above, a robust MCR model may be developed using numerous tissue measurements during the MCR modeling process, allowing the decay response components to be more specific or unique to the tissue types of interest. Generally, as discussed above, a training set of known tissue samples is analyzed using MCR to determine the multi-channel decay response components (equation 3). The analysis will concurrently determine the intensity values (equation 2) of each tissue sample, and a classification model can be prepared accordingly. Once the classification model is determined, forward looking classification of like tissue types may be performed by using MCR, ACLS, or CLS and applying the classification model to the resulting intensity values from that process.

The analysis of like tissue types may be performed in one of three ways:

(1) Continue to use MCR with the new measurement(s). According to some embodiments, the dataset will consist of a subset of the original training set combined with the new measurement(s). The original training subset plus the new measurement(s) would help delineate changes in the instrumental noise components (peak location, baseline artifacts, etc.). The main advantage with this approach is the ability to adapt and change when there are changes in the instrument noise. In this case, the main tissue components would be equality constrained along with the noise components, and the remaining components would have the ability to change and adapt. The MCR intensities from the main tissue components would determine the tissue classification.

(2) Classical Least Squares (CLS) approach. This approach uses equation 2 to obtain the intensities from a known set of pure decay response components. In this case, it would use the decay response components determined by MCR for future intensity (C) predictions. The CLS intensities from the main tissue components would determine the tissue classification.

(3) Augmented Classical Least Squares (ACLS) approach. This approach also uses equation 2 to obtain the intensities from a known set of pure decay response components. However, in this case the components describing the instrumental noise could be modified from the original MCR components, to reflect the most current noise sources. These noise sources are often determined with the use of a repeat sample taken over time. The ACLS intensities from the main tissue components would determine the tissue classification.

Each of these embodiments and obvious variations thereof is contemplated as falling within the spirit and scope of the present disclosure. Moreover, the present concepts expressly include any and all combinations and subcombinations of the preceding elements and aspects.

To provide aspects of the present disclosure, embodiments may employ any number of programmable processing devices that execute software or stored instructions. Physical processors and/or machines employed by embodiments of the present disclosure for any processing or evaluation may include one or more networked (Internet, cloud, WAN, LAN, satellite, wired or wireless (RF, cellular, WiFi, Bluetooth, etc.)) or non-networked general purpose computer systems, microprocessors, field programmable gate arrays (FPGAs), digital signal processors (DSPs), micro-controllers, smart devices (e.g., smart phones), computer tablets, handheld computers, and the like, programmed according to the teachings of the exemplary embodiments. In addition, the devices and subsystems of the exemplary embodiments can be implemented by the preparation of application-specific integrated circuits (ASICs) or by interconnecting an appropriate network of conventional component circuits. Thus, the exemplary embodiments are not limited to any specific combination of hardware circuitry and/or software.

Stored on any one or on a combination of computer readable media, the exemplary embodiments of the present disclosure may include software for controlling the devices and subsystems of the exemplary embodiments, for driving the devices and subsystems of the exemplary embodiments, for enabling the devices and subsystems of the exemplary embodiments to interact with a human user, and the like. Such software can include, but is not limited to, device drivers, firmware, operating systems, development tools, applications software, database management software, and the like. Computer code devices of the exemplary embodiments can include any suitable interpretable or executable code mechanism, including but not limited to scripts, interpretable programs, dynamic link libraries (DLLs), Java classes and applets, complete executable programs, and the like. Moreover, processing capabilities may be distributed across multiple processors for better performance, reliability, cost, or other benefit.

Common forms of computer-readable media may include, for example, a floppy disk, a flexible disk, hard disk, magnetic tape, any other suitable magnetic medium, a CD-ROM, CDRW, DVD, any other suitable optical medium, punch cards, paper tape, optical mark sheets, any other suitable physical medium with patterns of holes or other optically recognizable indicia, a RAM, a PROM, an EPROM, a FLASH-EPROM, any other suitable memory chip or cartridge, a carrier wave or any other suitable medium from which a computer can read. Such storage media can also be employed to store other types of data, e.g., data organized in a database, for access, processing, and communication by the processing devices.

### EXAMPLES

The following examples are not intended to limit the scope of the claims to the invention, but are rather intended to be exemplary of certain embodiments. Any variations in the exemplified methods which occur to the skilled artisan are intended to fall within the scope of the present invention.

### Example 1: Determining Component Signatures and Corresponding Intensities for Known (Training) Data Set

The researchers at Cedars-Sinai Medical Center collected TR-LIFS data from seven subjects. 35 measurements were collected from those seven subjects. Normal cortex, white matter and glioblastoma tissue regions were the main tissues investigated in this study. Each measurement consisted of exciting a tissue region within the brain with a 337 nm pulsed laser and collecting the fluorescence emission in a time-resolved manner [1]. Emission lifetime decay curves were collected at six different binned wavelength regions: 370-415 nm, 415-450 nm, 450-480 nm, 480-560 nm, 570-610 nm, and 610-800 nm. Excited fluorophores, specific to the tissue types of interest, may have more intense emissions at different wavelengths; therefore, collecting these fluorescence decay curves over six different wavelength bins should allow tissue discrimination to be more specific.

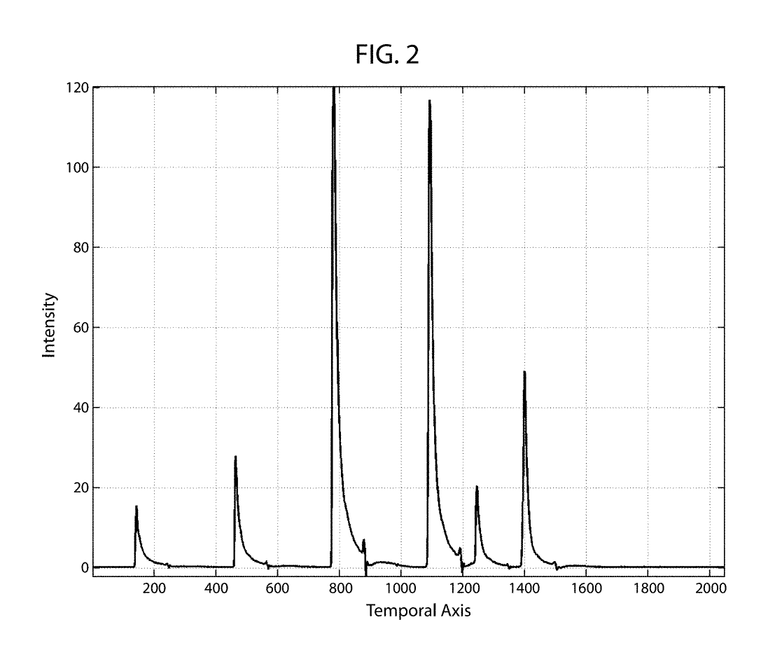

The preprocessing of the data consisted of the following. Each raw measurement consisted of 1000 repetitions of the 2048 temporal data points comprising the fluorescence decay response. These 1000 repetitions were averaged together to improve the overall signal to noise of the measurement. These 2048 temporal data points contain the decay curves for all six emission wavelength bins. FIG. 2 shows the measurement decay responses after the 1000 repetitions have been averaged together.

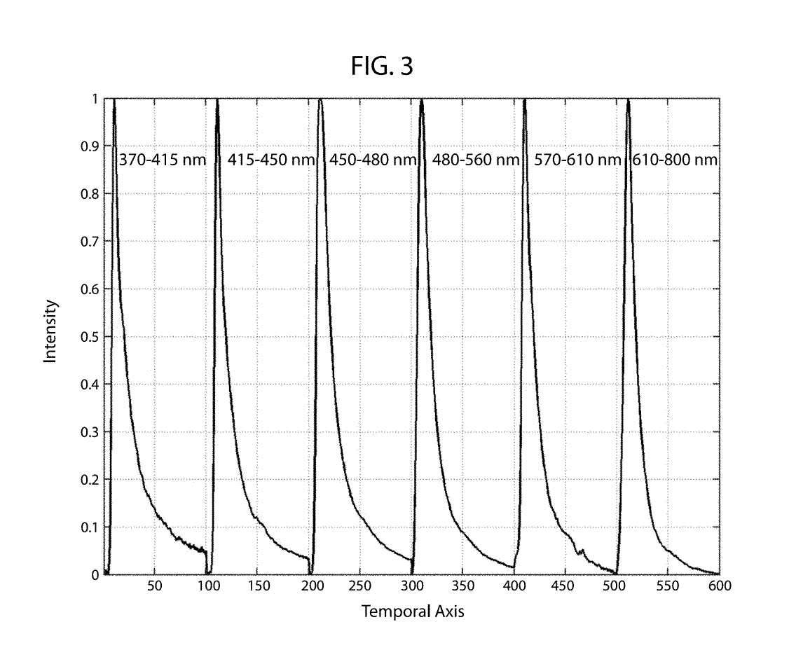

To focus on the decay responses of the wavelength bins, only temporal points about the peak for each decay curve were used. This was accomplished by including 10 temporal points prior to the start of the peak then extending for 100 points. Since the peak intensity could be affected by laser intensity and instrumental changes, the overall intensity was adjusted and normalized on a per decay curve basis. After the truncation of each decay curve, the minimum value of each decay curve was subtracted from the entire curve and then normalized by dividing by the maximum intensity. These decay curves were then concatenated back together to provide multi-channel fluorescence decay response curves for each measurement. Multi-channel refers to the six binned fluorescence emission channels. FIG. 3 shows the results of the measurement in FIG. 2 following these preprocessing treatments.

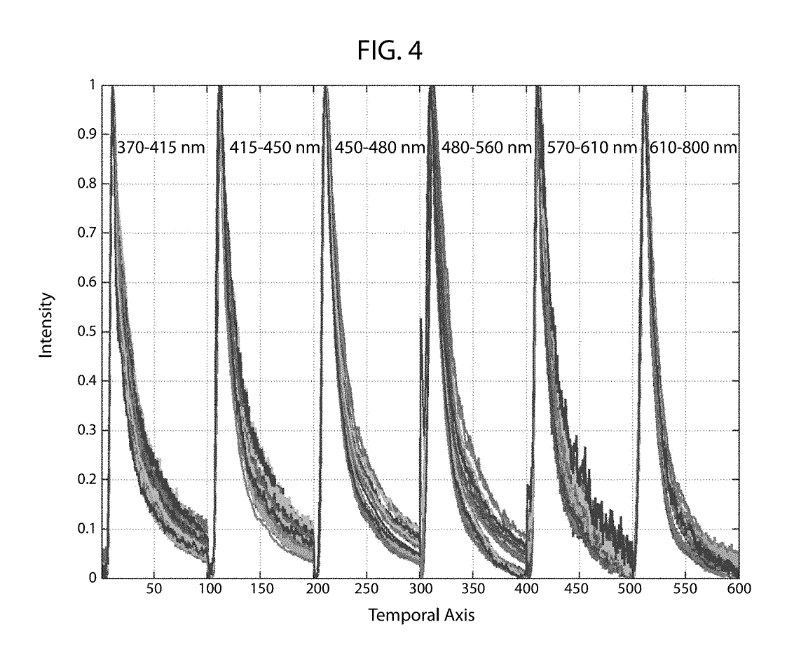

After all the data were preprocessed as described above, the data was combined together, so that the data can be analyzed using MCR to identify the differences in all six decay responses with respect to the normal, white matter and glioblastoma brain tissue. FIG. 4 shows all 35 measurements combined together after the truncation, normalization and concatenation step.

For the MCR analysis of these 35 measurements (FIG. 4), a mixture of non-negativity and equality constraints were used. The best results were obtained when initializing the MCR analysis with 17 decay response components. 11 of the components were chosen based upon the number of eigenvalues above the noise floor using a PCA Scree plot, and another 6 were included to model a specific saw-tooth noise pattern present in the data.

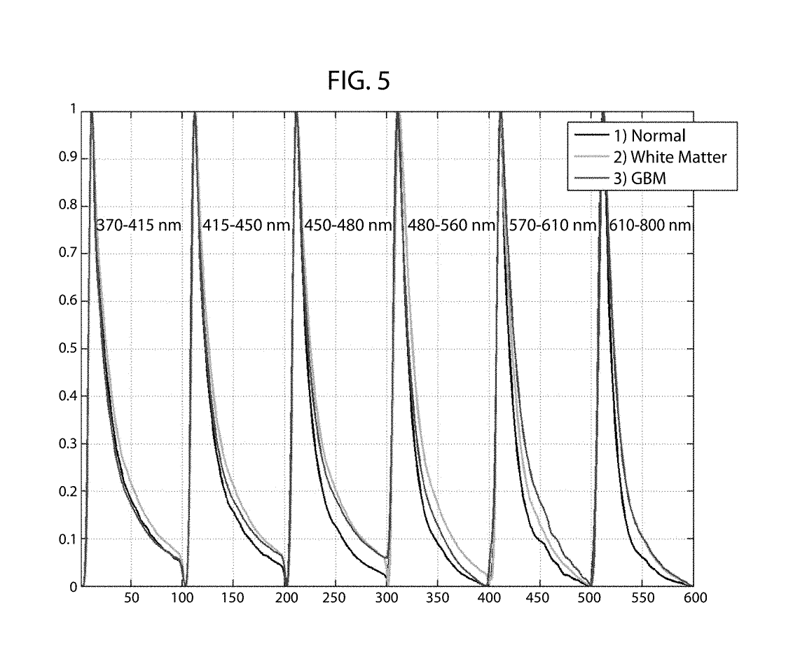

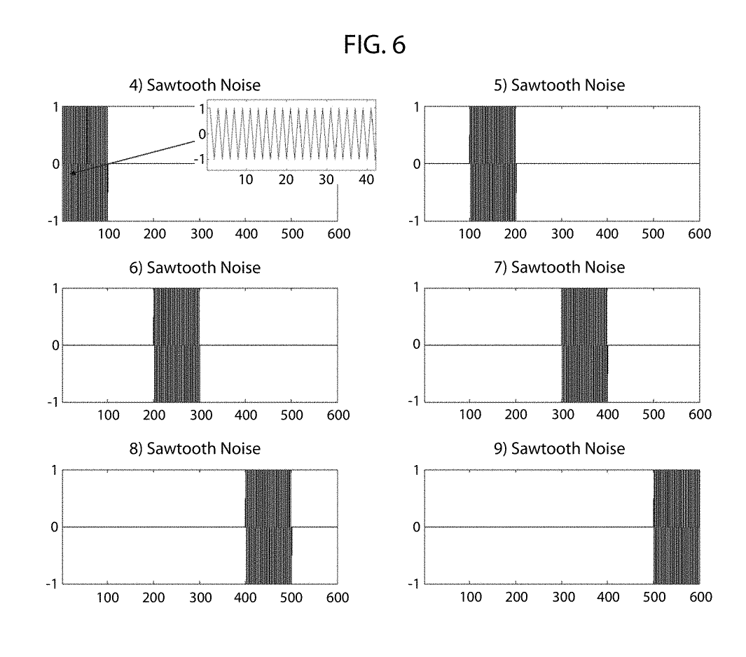

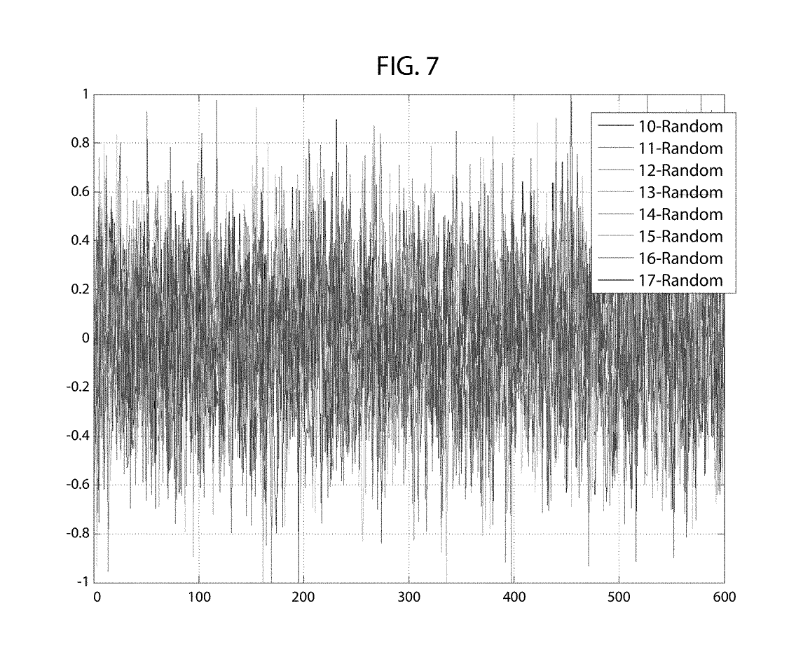

As described earlier, initial estimates of the decay response components is necessary to start the MCR analysis. These initial estimates can be from previous analyses, random numbers when nothing is known about the data set, or can be obtained using knowledge about the data set itself, or a combination of the above. Using the knowledge about which measurements were obtained from each tissue type, the measurements of like-tissue types were averaged together to obtain the initial starting components for the 3 tissue types (normal, white matter and glioblastoma (GBM)). See FIG. 5. In addition to the averaging, a Savitzky-Golay smooth was also used to smooth the noise from these tissue decay response components. After looking carefully at the data, it was observed that there was a correlated saw-tooth noise source present in the data. Therefore, a saw-tooth noise component was generated for each binned wavelength channel. The magnitude of this noise component changed depending on the binned wavelengths and measurement. This resulted in a total of 6 components. See FIG. 6. The remaining 8 components were initialized for MCR with random numbers. See FIG. 7.

FIG. 5 shows that there is a unique pattern across the multi-channel response curve for each tissue type. These however are the starting components and there is potential for MCR to change each one of these decay response curves depending on the best fit of the data. The components in FIG. 5 had non-negativity constraints placed upon them since there should not be negative fluorescence intensities. FIG. 6 shows the saw-tooth noise components. As mentioned earlier, since the amount of this noise varies depending on the binned wavelengths and measurement, it was decided to have six components, so that they can model each binned wavelength channel independently. These six components were equality constrained and the non-negativity constraint was removed. The last 8 MCR components were initialized using random numbers because there was no knowledge about these components. The non-negativity constraint was removed since it is expected that these components will model the small anomalies in the overall decay response.

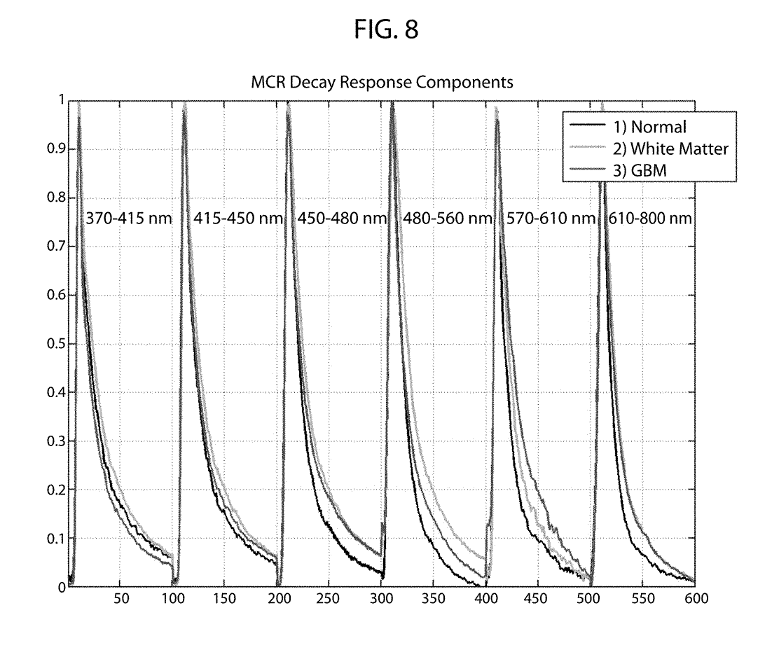

Application of the MCR analysis as described above developed the linear independent decay response components for each brain tissue type and their corresponding intensities. In addition, MCR modeled the other 14 components that accounted for noise and other measurement variations (peak location, baseline variation, etc.). FIG. 8 shows the final MCR decay response components related to the 3 tissue types.

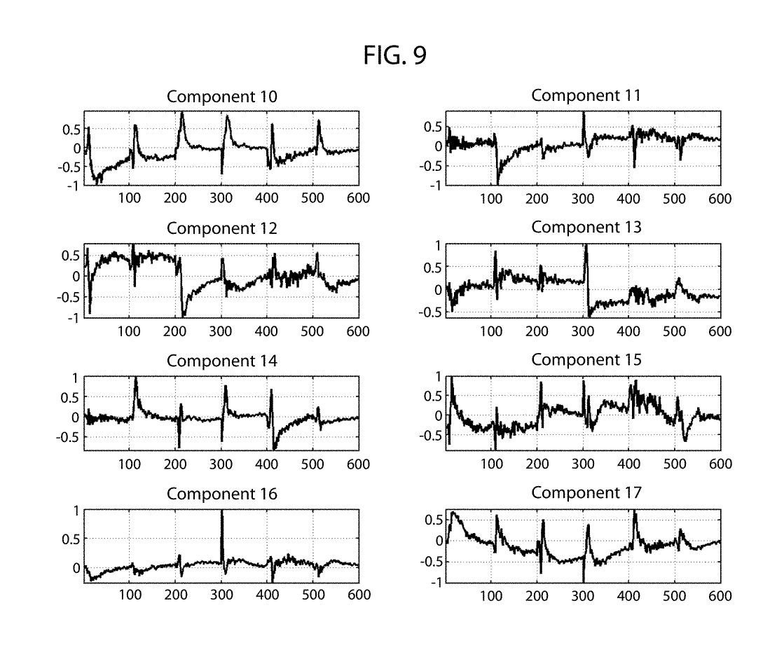

These components in FIG. 8 look similar to the starting components with slight variations. These components were modified by the MCR process to provide the best least squares fit of the data. These components model approximately 96% of the total decay response variance present in this data set. FIG. 9 shows the eight other components that model measurement related variance (peak location, baseline variation, etc.).

FIG. 10 shows the corresponding intensities (C) for the three tissue decay response components. Recall that both the decay response components (K) and intensities (C) are generated during the MCR process (see equations 2 and 3). As shown in FIG. 10, the intensities of the three main components facilitate the classification of tissue type due to the class separation of the intensity values. A model can be applied to classify future intensities obtained from this MCR model as long as the 3 main decay response components remain the same. The classification by intensity values can be applied to additional tissue samples using a discriminate model if desired. Examples of discriminate analysis methodologies that may be used for this purpose are: 1) Linear Discriminate Analysis (LDA), 2) Quadratic Discriminate Analysis (QDA) or 3) using Mahalanobis distance to discriminate.

FIG. 11 refers back to equation 1a, in which a single multi-channel response measurement is reconstructed using the MCR decay response components. The top of this figure shows the raw data (magenta) with the MCR reconstructed decay response curve overlaid (black). Notice the MCR fit is very good with only a small residual remaining (bottom plot). Also, since this particular measurement shown here (normal cortex measurement #4) is from normal tissue, component 1 is the most dominant component of all 17. It also shows that the next largest source of variance is located around the peak of each decay response for each binned wavelength channel.

### Example 2: Forward Looking Prediction of Tissue Type

In the above example, the decay curves were normalized to unity during the preprocessing step of the raw data, and the multi-channel decay components are also normalized to unity (unit intensity or one), therefore the intensity values for each of the 3 tissue components vary approximately from 0 to 1. Intensity values closer to one for one of the three tissue components would necessarily mean the other two components have to be closer to 0 because of the additive nature of the components (equation 1a). For example, from the current training data, a sample has the following intensity values:

i. Normal cortex multi-channel component intensity value=0.8

ii. White mater multi-channel component intensity value=0.12

iii. Glioblastoma multi-channel component intensity value=0.05

These values add up to 0.99, therefore the other values, such as noise, must make up the remaining 0.01, as the total sum should be approximately 1. Thus, this normal tissue sample is easily classified as such based on the high normal cortex multi-channel component intensity value.

In this example, review of the intensity values for the 35 samples shows that if the intensity of one of the components is greater than 0.51, then that component would have to determine the major tissue type present in the measurement (see also FIG. 10). For this training set, the grouping is well defined, as the sample closest to the boundary condition is a normal sample with the following values:

i. Normal cortex multi-channel component intensity value=0.57

ii. White mater multi-channel component intensity value=0.17

iii. Glioblastoma multi-channel component intensity value=0.23

These values add up to 0.97, therefore the other values, such as noise, must make up the other 0.03 so that the total sum is approximately 1. But it is still obvious from the intensity data that the normal cortex is the dominate signal present.

Thus, additional brain tissue measurements can be analyzed (using either MCR, CLS, or ACLS as described above) and classified according to this framework, to predict whether the tissue is normal cortex, white matter, or glioblastoma.

Additionally, according to some embodiments, the analysis may be performed on samples of mixed tissue type (for example, tissue samples having a portion of white matter and a portion of glioblastoma). A mixed tissue sample can be evaluated once the component signatures have been determined, using the process disclosed herein, as the analysis is able to determine how much of each component signature is present in the sample. Thus, the intensity value information may provide valuable insights into tissue composition even in the case where there is no majority component identified.

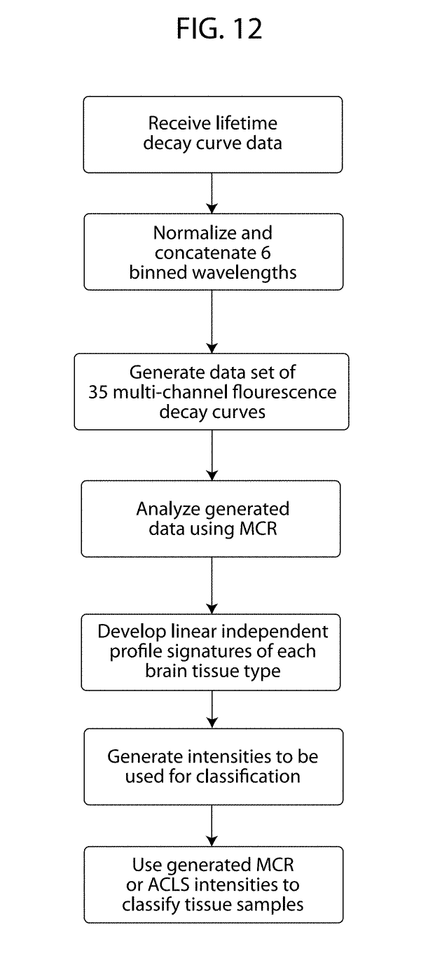

The process of determining component signatures and corresponding intensities for a known data set of brain tissue decay curve data, and using that determined information to classify additional brain tissue, as discussed in examples 1 and 2, is shown in FIG. 12.

The various methods and techniques described above provide a number of ways to carry out the application. Of course, it is to be understood that not necessarily all objectives or advantages described can be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as taught or suggested herein. A variety of alternatives are mentioned herein. It is to be understood that some preferred embodiments specifically include one, another, or several features, while others specifically exclude one, another, or several features, while still others mitigate a particular feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be employed in various combinations by one of ordinary skill in this art to perform methods in accordance with the principles described herein. Among the various elements, features, and steps some will be specifically included and others specifically excluded in diverse embodiments.

Although the application has been disclosed in the context of certain embodiments and examples, it will be understood by those skilled in the art that the embodiments of the application extend beyond the specifically disclosed embodiments to other alternative embodiments and/or uses and modifications and equivalents thereof.

Preferred embodiments of this application are described herein, including the best mode known to the inventors for carrying out the application. Variations on those preferred embodiments will become apparent to those of ordinary skill in the art upon reading the foregoing description. It is contemplated that skilled artisans can employ such variations as appropriate, and the application can be practiced otherwise than specifically described herein. Accordingly, many embodiments of this application include all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

All patents, patent applications, publications of patent applications, and other material, such as articles, books, specifications, publications, documents, things, and/or the like, referenced herein are hereby incorporated herein by this reference in their entirety for all purposes, excepting any prosecution file history associated with same, any of same that is inconsistent with or in conflict with the present document, or any of same that may have a limiting affect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

It is to be understood that the embodiments of the application disclosed herein are illustrative of the principles of the embodiments of the application. Other modifications that can be employed can be within the scope of the application. Thus, by way of example, but not of limitation, alternative configurations of the embodiments of the application can be utilized in accordance with the teachings herein. Accordingly, embodiments of the present application are not limited to that precisely as shown and described.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention.

### REFERENCES

1) Pramod V. Butte, Adam N. Mamelak, Miriam Nuno, Serguei I. Bannykh, Keith L. Black, Laura Marcu, Fluorescence lifetime spectroscopy for guided therapy of brain tumors, NeuroImage, Volume 54, Supplement 1, January 2011, Pages S125-S135, ISSN 1053-8119, (http://dx.doi.org/10.1016/j.neuroimage.2010.11.001).
2) Haaland D M, Jones H D T, Van Benthem M H, Sinclair M B, Melgaard D K, Stork C L, Pedroso M C, Liu P, Brasier A R, Andrews N L et al: Hyperspectral Confocal Fluorescence Imaging: Exploring Alternative Multivariate Curve Resolution Approaches. Appl Spectrosc 2009, 63(3):271-279.
3) Gallagher N B, Shaver J M, Martin E B, Morris J, Wise B M, Windig W: Curve resolution for multivariate images with applications to TOF-SIMS and Raman. Chemometrics and Intelligent Laboratory Systems 2004, 73(1):105-117.
4) Edward V. Thomas and David M. Haaland:Comparison of multivariate calibration methods for quantitative spectral analysis. Analytical Chemistry 1990 62 (10), 1091-1099
5) Edward V. Thomas: A primer on multivariate calibration. Analytical Chemistry 1994 66 (15), 795A-804A.
6) Haaland, David M., and David K. Melgaard. “New prediction-augmented classical least-squares (PACLS) methods: application to unmodeled interferents.” Applied Spectroscopy 54.9 (2000): 1303-1312.
7) Haaland, David M., and David K. Melgaard. “New augmented classical least squares methods for improved quantitative spectral analyses.” Vibrational Spectroscopy 29.1 (2002): 171-175.

What is claimed is:

1. A method for analysis of tissue, comprising:
applying time resolved laser induced fluorescence spectroscopy to a tissue, to measure lifetime time decay profile data relating to the tissue, wherein the lifetime time decay profile data is measured at a plurality of specific emission wavelength bands;
normalizing the lifetime time decay profile data for each of the plurality of specific emission wavelength bands;
concatenating the normalized lifetime time decay profile data for each of the plurality of specific emission wavelength bands, to generate a multi-channel fluorescence decay response curve;
applying multivariate curve resolution to the generated multi-channel fluorescence decay response curve, to generate a plurality of decay response signature components across the plurality of specific emission wavelength bands and corresponding intensity data;
performing a biopsy of the tissue to generate biopsy data;
determining, using the biopsy data and the intensity data, a tissue classification type indicated by the intensity data.

2. The method of claim 1, further comprising:
applying the method of claim 1 to a plurality of tissues, to generate a database of known classification data, the known classification data correlating the intensity data and the tissue classification type for each of the plurality of tissues.

3. The method of claim 2, further comprising:
applying time resolved laser induced fluorescence spectroscopy to a second tissue, to measure lifetime time decay profile data relating to the second tissue, wherein the lifetime time decay profile data is measured at a plurality of specific emission wavelength bands;
normalizing the lifetime time decay profile data of the second tissue for each of the plurality of specific emission wavelength bands;
concatenating the normalized lifetime time decay profile data for each of the plurality of specific emission wavelength bands, to generate a multi-channel fluorescence decay response curve;
applying least squares analysis to the generated multi-channel fluorescence decay response curve, for each of the specific emission wavelength bands, using the generated plurality of decay response signature components, to quantify the amount of each decay response signature component;
classifying the second tissue by comparing the amount of each decay response signature component to the database of known classification data.

4. The method of claim 1, wherein the plurality of specific emission wavelength bands comprise six specific wavelength bands.

5. The method of claim 4, wherein the six specific wavelength bands comprise 365-410 nanometers, 410-450 nanometers, 450-480 nanometers, 480-550 nanometers, 550-600 nanometers, and above 600 nanometers.

6. The method of claim 1, wherein the tissue is one of brain tissue, breast tissue, colon tissue, skin tissue, or lung tissue.

7. The method of claim 1, wherein the tissue is brain tissue, and the tissue classification type comprises normal cortex, white matter, necrotic tissue, or glioblastoma.

8. The method of claim 3, wherein the second tissue is living human tissue, and the method is applied to the second tissue during a surgical operation to classify the second tissue before completion of the surgical operation.

9. The method of claim 1, wherein the tissue is in vivo.

10. The method of claim 1, wherein the tissue is ex vivo.

11. The method of claim 3, wherein the least squares analysis comprises classical least squares analysis.

12. The method of claim 3, wherein the least squares analysis comprises augmented classical least squares analysis.

13. A system for diagnosis of human tissue, comprising:
a database of human tissue data comprising a plurality of tissue classification types and a plurality of decay profile signatures and corresponding intensities;
a scope for collecting time resolved laser induced fluorescence spectroscopy data from a human tissue;
a processor configured to receive time resolved laser induced fluorescence spectroscopy data from the scope, determine lifetime decay profile data from the time resolved laser induced fluorescence spectroscopy data, and generate decay profile signature data and corresponding intensity data based on the lifetime decay profile data;
wherein the processor communicates with the database to identify the tissue classification type according to the intensity data.

14. The system of claim 13, wherein the tissue is in vivo.

15. The system of claim 13, wherein the tissue is ex vivo.

16. The system of claim 13, wherein the decay profile signature data is determined at a plurality of specific emission wavelength bands.

17. The system of claim 16, wherein the plurality of specific emission wavelength bands comprise six specific wavelength bands.

18. The system of claim 17, wherein the six specific wavelength bands comprise 365-410 nanometers, 410-450 nanometers, 450-480 nanometers, 480-550 nanometers, 550-600 nanometers, and above 600 nanometers.

19. The system of claim 13 wherein the human tissue is brain tissue, and the plurality of tissue classification types comprise normal cortex, white matter, necrotic tissue, or glioblastoma.

20. A method for identifying human tissue according to spectral information, using a computing system, the computing system comprising one or more processors communicatively coupled to a network database, the method comprising:
applying time resolved laser induced fluorescence spectroscopy to the human tissue, to measure lifetime time decay profile data relating to the human tissue, wherein the lifetime time decay profile data is measured at a plurality of specific emission wavelength bands;
normalizing the lifetime time decay profile data for each of the plurality of specific emission wavelength bands;
concatenating the normalized lifetime time decay profile data for each of the plurality of specific emission wavelength bands, to generate a multi-channel fluorescence decay response curve;
applying a curve fitting technique, using the one or more processors, to the generated multi-channel fluorescence decay response curve, to determine intensity data corresponding to a plurality of decay response signature components;
sending, using the one or more processors, a request to the network database to identify the human tissue, the request containing information relating to at least one of the plurality of decay response signature components and corresponding intensity data;
receiving, from the network database, a response to the request, the response indicating the tissue classification type corresponding to the human tissue according to the intensity data.

21. The method of claim 20, wherein the human tissue is in vivo.

22. The method of claim 20, wherein the human tissue is ex vivo.

23. The method of claim 20, wherein the plurality of specific emission wavelength bands comprise six specific wavelength bands.

24. The method of claim 23, wherein the six specific wavelength bands comprise 365-410 nanometers, 410-450 nanometers, 450-480 nanometers, 480-550 nanometers, 550-600 nanometers, and above 600 nanometers.

25. The method of claim 20, wherein the one or more processors are communicatively coupled to the network database via the Internet.

26. The method of claim 20, wherein the one or more processors are communicatively coupled to the network database via a private secured network.

27. The method of claim 20, wherein the network database comprises classification data relating tissue classification types to intensity data, the classification data determined by analysis of a plurality of known tissue types.

28. The method of claim 20, wherein the curve fitting technique comprises multivariate curve resolution.

29. The method of claim 20, wherein the curve fitting technique comprises classical least squares analysis.

30. The method of claim 20, wherein the curve fitting technique comprises augmented classical least squares analysis.

31. The method of claim 20 wherein the human tissue is brain tissue, and the plurality of tissue classification types comprise normal cortex, white matter, necrotic tissue, or glioblastoma.
